(19) United States
(12) Patent Application Publication
(10) Pub. No.: US 2026/0041365 A1
(43) Pub. Date: Feb. 12, 2026

(54) MULTI-MODAL DATA ACQUISITION AND HYDROCEPHALUS DIAGNOSIS METHOD BASED ON CEREBROSPINAL FLUID CIRCULATION

(71) Applicant: HAIHE LABORATORY OF BRAIN-COMPUTER INTERACTION AND HUMAN-MACHINE INTEGRATION, Tianjin (CN)

(72) Inventors: Xiuyun LIU, Tianjin (CN); Dong MING, Tianjin (CN); Xinyang LIU, Tianjin (CN); Xiaoguang TONG, Tianjin (CN); Huijie YU, Tianjin (CN); Marek Czosnyka, Tianjin (CN); Zofia Czosnyka, Tianjin (CN); Peter Smielewski, Tianjin (CN); Meijun PANG, Tianjin (CN); Kuo ZHANG, Tianjin (CN)

(73) Assignee: HAIHE LABORATORY OF BRAIN-COMPUTER INTERACTION AND HUMAN-MACHINE INTEGRATION, Tianjin (CN)

(21) Appl. No.: 19/218,686

(22) Filed: May 27, 2025

(30) Foreign Application Priority Data
Aug. 12, 2024 (CN) 202411100397.1

(51) Int. Cl. A61B 5/00; A61B 5/0205; A61B 5/0245; A61B 5/03; A61B 5/145; A61B 5/318; G16H 15/00; G16H 50/20

(52) U.S. Cl. CPC A61B 5/4064; A61B 5/0205; A61B 5/0245; A61B 5/031; A61B 5/14542; A61B 5/318; A61B 5/7239; A61B 5/7264; G16H 15/00; G16H 50/20; A61B 2562/0247

(57) ABSTRACT

A multi-modal data acquisition and hydrocephalus diagnosis method based on CSF circulation is provided. Firstly, after performing a lumbar puncture for the patient, the lumbar puncture needle is connected to a pressure sensor and a micro pump, and then, the intracranial pressure baseline of the patent is collected for 5 min, the micro pump is started, and the patient is infused with normal saline at a rate of 1 cc/min until the patient's intracranial pressure reaches the ICP plateau, during the period, multiple physiological parameters such as intracranial pressure and heart rate are monitored; finally, the collected parameters such as intracranial pressure are substituted into the established physical model of CSF circulation, and the CSF kinetic parameters such as CSF absorption resistance are calculated for auxiliary diagnosis.

### CROSS REFERENCE TO THE RELATED APPLICATIONS

This application is based upon and claims priority to Chinese Patent Application No. 202411100397.1, filed on Aug. 12, 2024, the entire contents of which are incorporated herein by reference.

### TECHNICAL FIELD

The invention relates to the field of clinical engineering, in particular to a multi-modal data acquisition and hydrocephalus diagnosis method based on cerebrospinal fluid circulation.

### BACKGROUND

Hydrocephalus is a pathological condition caused by the accumulation of cerebrospinal fluid in the brain, it can lead to increased intracranial pressure in patients, resulting in gait disorders, cognitive dysfunction, urinary incontinence, and other symptoms, which seriously affect the everyday life of patients, if the treatment is not timely, it may lead to death. Idiopathic normal pressure hydrocephalus, as ‘reversible dementia’, can effectively improve clinical symptoms or even reverse through early diagnosis and treatment, it is only dementia that can be improved by surgical treatment. Therefore, how to diagnose hydrocephalus early and screen out hydrocephalus patients who can benefit from surgical treatment is a major problem that currently plagues clinicians.

At present, the diagnosis of patients with hydrocephalus mainly depends on clinical indicators such as imaging examination, gait assessment, and the results of lumbar puncture to release cerebrospinal fluid. However, clinical indicators such as imaging features and gait assessment are often similar to neurodegenerative diseases, and it is difficult to effectively diagnose and distinguish them. In addition, the lumbar puncture release cerebrospinal fluid diagnosis process takes a long time, it often takes three days to determine whether the patient has hydrocephalus, which greatly delays the patient's treatment time and increases the patient's economic burden. Therefore, early diagnosis is of great significance for timely intervention and treatment of hydrocephalus.

### SUMMARY

The purpose of the invention is to propose a multi-modal data acquisition and hydrocephalus diagnosis method based on cerebrospinal fluid circulation, which solves the problem that the traditional diagnosis method cannot effectively diagnose and distinguish, and reduces the damage caused by multiple cerebrospinal fluid drainage tests to patients.

In order to achieve the above purpose, the invention proposes a multi-modal data acquisition and hydrocephalus diagnosis method based on cerebrospinal fluid circulation, the steps are as follows:
S1, lumbar puncture;
S2, perfusion test;
S3, signal acquisition;
S4, data analysis.

Preferably, in S1, a puncture point is selected at an intersection of a posterior superior iliac spine line and a posterior median line, corresponding to a 4-5 space of the lumbar spinous process, after a skin of the puncture point is safely fixed, a lumbar puncture needle is inserted perpendicular to a back direction or slightly inclined to a head side.

Preferably, after performing a lumbar puncture for the patient, the lumbar puncture needle is connected to a pressure sensor and a micro pump, and an intracranial pressure value of the patient at this time is continuously recorded for 5 min, that is, the intracranial pressure (ICP) baseline.

Preferably, in S2, the micro pump is started, and the normal saline is injected into the lumbar intervertebral space at a constant speed of 60 mL/h, and the intracranial pressure value and various physiological indicators of a patient are recorded and monitored in real-time through intensive care monitoring plus (ICM+) until the intracranial pressure of the patient reaches a stable state, and the intracranial pressure value of the patient at this time is continuously recorded for 5 min, that is, ICP plateau.

Preferably, in S3, data acquisition is composed of a monitor, a medical pressure sensor, a blood oxygen probe, a five-lead electrocardiogram (ECG) monitoring, and an ICM+ multi-modal detection software.

Preferably, the medical pressure sensor is mainly used to measure intracranial pressure, the blood oxygen probe is mainly used to measure blood oxygen, the five-lead ECG monitoring is used to measure the patient's heart rate, the monitor and ICM+ multi-modal detection software are used to monitor and record the patient's data in real-time.

Preferably, in S4, the specific steps of the data analysis are as follows:
S41, establishing a cerebrospinal fluid (CSF) circulation model;
S42, calculating a CSF outflow resistance;
S43, calculating an intracranial pressure pulse amplitude (AMP) and elasticity coefficient (Elasicity);
S44, generating test reports for auxiliary diagnosis.

Preferably, in S41, intracranial compliance denotes a volume compensation function in the cranial cavity, the intracranial compliance is a volume change caused by a change of unit intracranial pressure, the calculation formula is as follows:

$$C = \frac{{dV}_{IC}}{{dP}_{IC}} \qquad ( 1 )$$

where C denotes the intracranial compliance, dV_{IC} denotes the change of intracranial volume, and dP_{IC} denotes the change of intracranial pressure;
when a volume of a cranial spinal cord gap increases, the compliance when the intracranial pressure is in a low-pressure state is much higher than that when the intracranial pressure is in a high-pressure state, the calculation formula of the intracranial compliance C is as follows:

$$C = \frac{1}{{KP}_{IC}} \qquad ( 2 )$$

where P_{IC} denotes the intracranial pressure, K denotes a specific elastic constant of a CSF circulatory system of the patient;
in a modeling of the CSF circulation, the calculation formula of intracranial compliance C is as follows:

$$C = \frac{1}{K ( {P}_{IC} - {P}_{0} )} \qquad ( 3 )$$

where P₀ denotes a CSF constant storage pressure;
a model establishment includes a whole circulation pathway of CSF in a human body, and also includes an infusion of normal saline, based on a law of conservation of liquid, a total inflow of the liquid is equal to its outflow, and the expression is as follows:

$${q}_{f} + {q}_{ex} = {q}_{a} + {q}_{s} \qquad ( 4 )$$

Where q_{f} denotes a rate of the CSF formation, qₑₓ denotes a rate of a normal saline perfusion in subsequent perfusion tests, qₐ denotes a CSF pathway for continuous circulation absorption, and qₛ denotes the CSF pathway stored in an intracranial support and protection of a brain;
a difference between the intracranial pressure and a dural venous sinus pressure P_{d} is calculated as follows:

$${q}_{a} = \frac{{P}_{IC} - {P}_{d}}{R} \qquad ( 5 )$$

where R denotes a CSF outflow resistance, P_{d} is the dural venous sinus pressure;
a change function of the intracranial pressure with time is obtained by substituting into Formula (1) and Formula (2):

$$\frac{{dP}_{IC} ( {V}_{IC} ( t ) )}{dt} = \frac{{dP}_{IC}}{{dV}_{IC}} \frac{{dV}_{IC}}{dt} = {KP}_{IC} \frac{{dV}_{IC}}{dt} \qquad ( 6 )$$

Where t denotes time, dt denotes a time change;
a cerebral blood flow is kept constant and a storage pathway of CSF is calculated, the formula is as follows:

$${q}_{s} = \frac{{dV}_{IC}}{dt} \qquad ( 7 )$$

Formula (6) and Formula (4) are combined, the calculation formula is as follows:

$$\frac{{dP}_{IC}}{dt} = {KP}_{IC} {q}_{s} = {KP}_{IC} ( {q}_{f} + {q}_{ex} - {q}_{a} ) \qquad ( 8 )$$

a steady-state intracranial resting pressure Pᵣ is regulated by the dural venous sinus pressure, a CSF outflow resistance R, and a CSF formation rate q_{f}, the calculation formula is as follows:

$${P}_{r} = {P}_{d} + {q}_{f} R \qquad ( 9 )$$

Formula (8) and Formula (5) are substituted into Formula (9), a nonlinear differential equation of the model is obtained:

$$\frac{{dP}_{IC} ( t )}{dt} + {\frac{K}{R} [ {P}_{IC} ( t ) ]}^{2} - ( {Kq}_{ex} ( t ) + \frac{{KP}_{r}}{R} ) {P}_{IC} ( t ) = 0 \qquad ( 10 )$$

in summary, a mathematical model of CSF circulation is established, and a relationship function between the intracranial pressure and the perfusion duration is constructed.

Preferably, in S42, an integral factor method is used to solve Formula (10), the formula is as follows:

$${P}_{IC} ( t ) = \frac{{e}^{\frac{K}{R} {\int }_{0}^{t} ( {Rq}_{ex} ( τ ) + {P}_{r} ) d τ}}{\frac{K}{R} {\int }_{0}^{t} {e}^{\frac{K}{R} {\int }_{0}^{t} ( {Rq}_{ex} ( τ ) + {P}_{r} ) d τ} + \frac{1}{{{P}_{IC}}^{( 0 )}}} \qquad ( 11 )$$

where τ is an independent variable in an integral process;
when the pressure gradually decreases from Pₚ to the resting pressure after P_{IC} rises to Pₚ through the perfusion test, the expression of a time relationship between the intracranial pressure and perfusion is as follows:

$${P}_{IC} ( t ) = \frac{{p}_{P} {e}^{\frac{Kt {P}_{r}}{R}}}{1 + \frac{{P}_{P}}{{p}_{r}} ( {e}^{\frac{{k}_{t} {P}_{r}}{R}} - 1 )} \qquad ( 12 )$$

where p_{P} is a value of the intracranial pressure rising to the platform period;
under the condition of resting pressure, P₀=0, the intracranial pressure P_{IC}(0) is a steady-state value, that is, Pᵣ, the perfusion speed is qₑₓ(t)=q_{inf}, the calculation formula of P_{IC}(t) is as follows:

$${P}_{IC} ( t ) = \frac{{P}_{r} ( {P}_{r} + {Rq}_{inf} )}{{P}_{r} + {Rq}_{inf} {e}^{- \frac{K}{R} ( {P}_{r} + {Rq}_{inf} ) t}} \qquad ( 13 )$$

where q_{inf} denotes the perfusion speed;
under the condition of P₀≠0, the calculation formula of P_{IC}(t) is as follows:

$${P}_{IC} ( t ) = \frac{( {q}_{inf} + \frac{{P}_{r} - {P}_{0}}{R} ) ( {P}_{r} - {P}_{0} )}{\frac{{P}_{r} - {P}_{0}}{R} + {q}_{inf} [ {e}^{- K ( \frac{{P}_{r} - {P}_{0}}{R} + {q}_{inf} ) t} ]} + {P}_{0}$$

in the process of a constant pressure infusion test, dynamic data from resting state to steady state are collected and calculated, for the constant pressure infusion under a steady state condition, the differential equation can be transformed into:

$${q}_{inf} = \frac{{P}_{IC} - {P}_{r}}{R} \qquad ( 15 )$$

the CSF outflow resistance parameter R is solved, and the calculation formula is as follows:

$$R = \frac{{P}_{level} - {P}_{r}}{{q}_{inf}} \qquad ( 16 )$$

where Pₗₑᵥₑₗ is a steady-state intracranial pressure produced by the constant pressure perfusion.

Preferably, in S43, a frequency domain analysis and extraction of the intracranial pressure is carried out to obtain the intracranial pressure pulse amplitude, it is considered that the intracranial pressure pulse amplitude can reflect the increase of intracranial pressure within a certain range, at the same time, the elasticity coefficient Elasicity is used to represent the brain compliance, when Elasicity is low, the brain compliance is poor.

Therefore, the invention proposes a multi-modal data acquisition and hydrocephalus diagnosis method based on CSF circulation, and its beneficial effects are as follows:
(1) The invention proposes to calculate the outflow resistance of patients by monitoring various physiological parameters such as intracranial pressure during lumbar puncture and drainage, which can effectively improve the diagnostic accuracy of hydrocephalus, greatly shorten the diagnosis time of patients, and reduce the occurrence of misdiagnosis and missed diagnosis.
(2) The invention effectively reduces the economic burden of patients and the social burden of hospitals, at the same time, the technology can help doctors better understand the pathogenesis of hydrocephalus diseases, so as to explore more effective treatment methods.
(3) The invention is expected to be applied to the diagnosis of related diseases such as congenital hydrocephalus, traumatic and post-subarachnoid hemorrhage-induced hydrocephalus, and the formulation of personalized treatment strategies.

The following is a further detailed description of the technical scheme of the invention through drawings and embodiments.

### BRIEF DESCRIPTION OF THE DRAWINGS

FIG. 1 is an experimental flow chart of the multi-modal data acquisition and hydrocephalus diagnosis method based on CSF circulation.

FIG. 2 is a schematic diagram of the perfusion test of a multi-modal data acquisition and hydrocephalus diagnosis method based on CSF circulation.

FIG. 3 is an equivalent circuit diagram of the CSF circulation dynamics modeling of the multi-modal data acquisition and hydrocephalus diagnosis method based on CSF circulation.

FIGS. 4A-4F are schematic diagrams of the difference analysis of various physical indexes of CSF circulation dynamics of the multi-modal data acquisition and hydrocephalus diagnosis method based on CSF circulation.

### DETAILED DESCRIPTION OF THE EMBODIMENTS

The following is a further detailed description of the technical scheme of the invention through drawings and embodiments.

Unless otherwise defined, the technical terms or scientific terms used in the invention should be understood by people with general skills in the field to which the invention belongs.

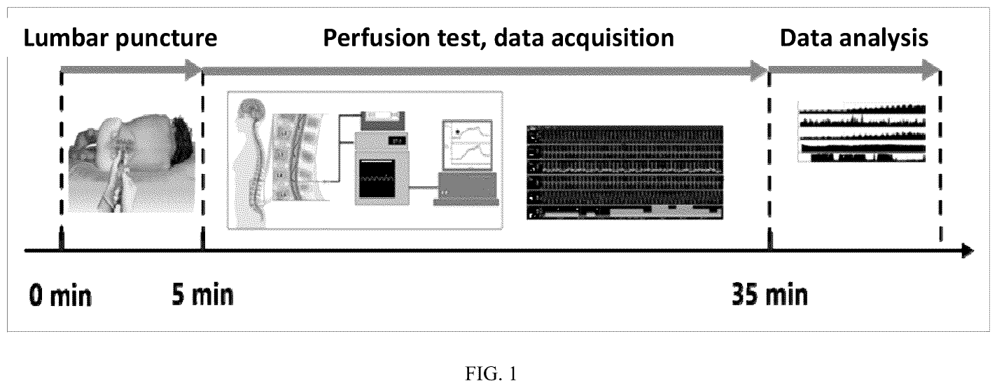

As shown in FIG. 1, the overall design of the invention mainly includes a lumbar puncture, a perfusion test, signal acquisition, and data processing. The lumbar puncture needle of the lumbar puncture adopts the model of Quincke No. 12 18G (the external diameter is 1.27 mm). The perfusion test adopts a micro pump to inject normal saline into the lumbar intervertebral space at a speed of 60 mL/h. The data acquisition is composed of a GE Dash 4000 monitor, a medical pressure sensor, a blood oxygen probe, a five-lead ECG monitoring, and an ICM+ multi-modal detection software, which can monitor and record the physiological indexes of patients during the perfusion test in real-time. The medical pressure sensor is mainly used to measure the intracranial pressure. The blood oxygen probe is mainly used to measure blood oxygen. The five-lead ECG monitor is used to measure the patient's heart rate, and the GE Dash 4000 monitor and ICM+ software are used to monitor and record the patient's data in real-time. The main function of the data processing module is to put the data recorded by ICM+ into the established CSF circulation model, and calculate the CSF outflow resistance, intracranial pressure pulse amplitude, and other CSF dynamics-related parameters, so as to provide more accurate diagnostic criteria for doctors.

After the patient is admitted to the hospital for examination, it is confirmed that there is no contraindication for lumbar puncture, the patient needs to lie on the bed on the curved side, hold the knees with both hands, and highlight the lumbar vertebral body and its spaces. After strict implementation of aseptic operation and disinfection procedures, the puncture point is selected. The puncture point is selected at the intersection of the posterior superior iliac spine line and the posterior median line, which usually corresponds to the 4-5 space of the lumbar spine. After the skin of the puncture point is securely fixed, the puncture needle is inserted perpendicular to the back direction or slightly inclined to the head side. When the needle passes through the ligament, the doctor will feel a significant sense of resistance disappearance. At this time, the needle core is gently pulled out, and it can be observed that the clear CSF flows out along the lumbar puncture needle.

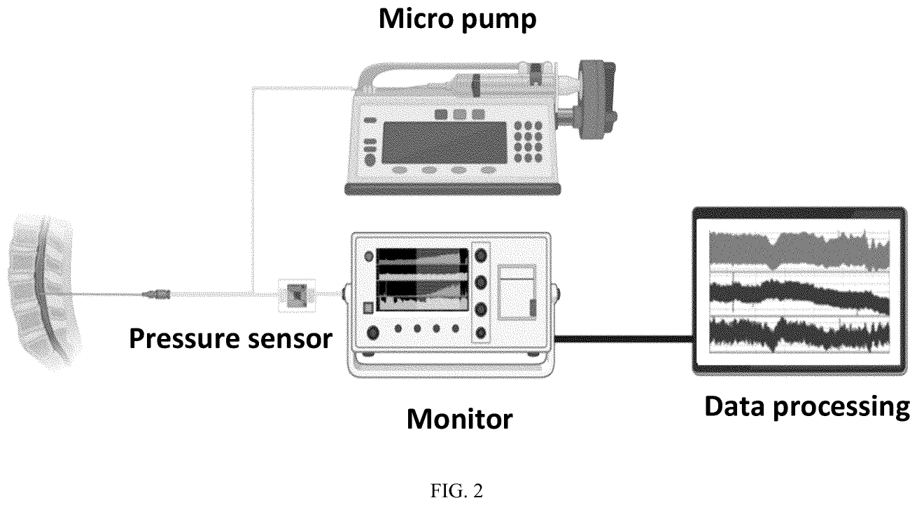

As shown in FIG. 2, after the lumbar puncture is completed, the pressure sensor is connected to the puncture needle, and the intracranial pressure value of the patient at this time is continuously recorded for 5 min, that is, the intracranial pressure baseline (ICP baseline). After the baseline record is completed, the micro pump is opened and normal saline is injected into the lumbar intervertebral space at a constant speed of 60 mL/h. At this time, the patient's intracranial pressure will slowly increase, in this process, the medical pressure sensor is mainly used to measure the intracranial pressure. The blood oxygen probe is mainly used to measure blood oxygen. The five-lead ECG monitor is used to measure the patient's heart rate. The GE Dash 4000 monitor and ICM+ software are used to monitor and record the patient's data in real-time. Until the patient's intracranial pressure reaches a steady state, the intracranial pressure values are recorded continuously at this time of 5 min, that is, the ICP plateau.

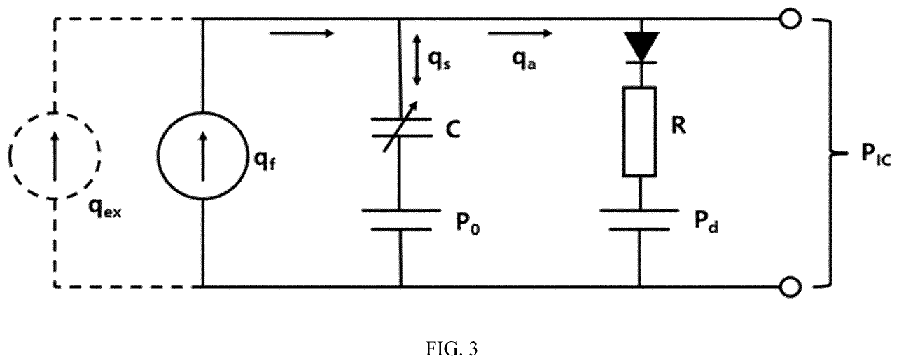

As shown in FIG. 3, according to the relevant literature, a normal adult can produce about 500 mL of CSF a day, which is regarded as a constant current source q_{f}. After CSF is produced, a part of CSF is stored in the intracranial cavity to support and protect the brain, the pathway of this part is set as qₛ, and at the same time, the pressure stored in the CSF is quantified as a voltage source, it is set as P₀, because there is a compensatory mechanism in the CSF circulation, a variable capacitance C is added to represent the pressure of the compensatory part; the pathway that the other CSF continues to circulate and absorb the pathway is set to be qₐ, the absorbed CSF circulates into the venous blood after the dural venous sinus, and the intracranial pressure P_{IC} is equivalent to the dural venous sinus pressure P_{d} and pressure resistance R. qₑₓ is a constant value, it denotes the rate of saline perfusion in subsequent perfusion tests. At the same time, it is assumed that the rate of perfusion is proportional to the pressure gradient on the side of the dural sinus, so the resistance R is used to represent the pressure difference between the intracranial pressure and the dural sinus. At the same time, the CSF circulation is directional, and a diode is added to the established model to represent the direction of the CSF circulation.

The multi-modal physiological parameters such as intracranial pressure collected during the perfusion test are substituted into the CSF model established in the data analysis to obtain a variety of CSF kinetic parameters and generate diagnostic reports for doctors and patients. Doctors can make auxiliary diagnoses according to these parameters of patients. As shown in Table 1, the outflow resistance Rcsf of patient A is 17.90 mmHg*min/mL, and the shunt operation will have great benefits, while the outflow resistance Rcsf of patient B is 9.73 mmHg*min/mL, and the benefit of shunt operation is very small, which is not suitable for shunt operation, and may be considered as other neurological diseases.

TABLE 1
Diagnosis report of CSF infusion test

| A | | | | B | | | |
|---|---|---|---|---|---|---|---|
| Infusion rate | 1.00 | Normalised | 2.946 | Infusion rate | 1 | Normalised | 1.437 |
| [mL/min] | | Error[%] | | [mL/min] | | Error[%] | |
| One needle | yes | Calculation | static | One needle | yes | Calculation | dynamic |
| only | | mode | | only | | mode | |
| ICP baseline | 3.82 | ICP plateau | 21.72 | ICP baseline | 2.44 | ICP plateau | 12.16 |
| [mmHg] | | [mmHg] | | [mmHg] | | [mmHg] | |
| AMP | 0.20 | AMP plateau | 3.18 | AMP | 0.34 | AMP plateau | 1.58 |
| baseline[mmHg] | | [mmHg] | | baseline[mmHg] | | [mmHg] | |
| Rcsf | 17.90 | Elasticity | 0.31 | Rcsf | 9.73 | Elasticity | 0.39 |
| [mmHg*min/mL] | | [1/mL] | | [mmHg*min/mL] | | [1/mL] | |
| PVI[mL] | 7.49 | Pss[mmHg] | −2.09 | PVI[mL] | 5.97 | Pss[mmHg] | 0.78 |
| CSF | 0.33 | Volume | 18.6 | CSF | 0.17 | Volume | 19.07 |
| production | | infused [mL] | 1 | production | | infused [mL] | |
| rate | | | | rate | | | |
| [mL/min] | | | | [mL/min] | | | |
| infusion | 18.61 | Needle resist. | 0.00 | infusion | 19.07 | Needle resist. | 0.44 |
| duration | | [mmHg*min/mL] | | duration | | [mmHg*min/mL] | |
| [min] | | | | [min] | | | |

Under the condition of high compliance, the intracranial pressure pulse amplitude caused by volume change is small; however, when the compliance decreases, the intracranial pressure pulse amplitude increases significantly. Therefore, it is considered that the intracranial pressure pulse amplitude can reflect the increase of intracranial pressure within a certain range. At the same time, we use the elasticity coefficient Elasicity to represent brain compliance. When the Elasicity is low, it indicates that brain compliance is poor, and vascular lesions may occur, providing new diagnostic ideas and methods for doctors.

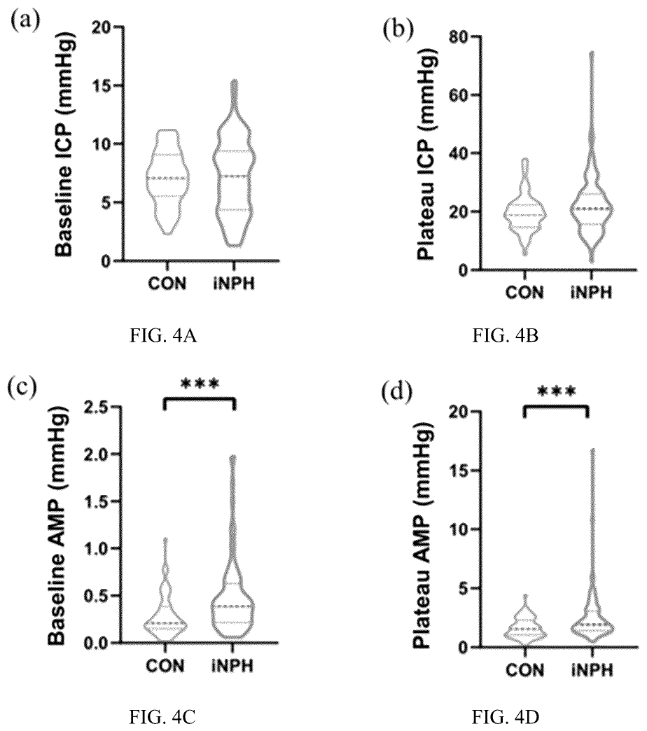

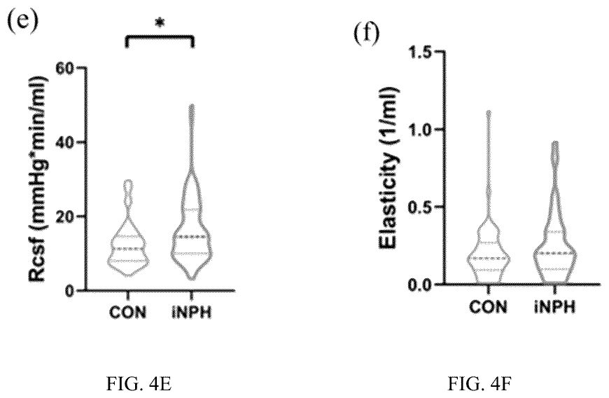

As shown in FIGS. 4A-4F, by collecting and analyzing the ICP baseline, ICP plateau, AMP baseline, AMP plateau, outflow resistance, and Elasicity of 131 patients in the control group (n=49) and patients with idiopathic normal pressure hydrocephalus (n=82), it is found that AMP baseline (Mean±SD, 0.29±0.22 vs. 0.48±0.39, p=0.001), AMP plateau (1.67±0.87 vs. 2.57±2.19, p=0.001), and CSF outflow resistance (12.44±6.01 vs. 17.75±15.42, p=0.024) are significantly different, indicating that AMP baseline, AMP plateau, and CSF outflow resistance can be used as diagnostic markers for idiopathic normal pressure hydrocephalus to help doctors perform auxiliary analysis.

Subsequently, ROC analysis is performed on these data (Area Under Curve, AUC), that is, the area under the curve. The critical point on the ROC curve denotes the critical value between the diseased group and the control group. By calculation, when the AMP baseline is greater than 0.33 mmHg, the AMP plateau is greater than 1.21 mmHg, or CSF outflow resistance is greater than 14.08 mmHg*min/mL, the patient can be diagnosed with idiopathic normal pressure hydrocephalus.

Therefore, a multi-modal data acquisition and hydrocephalus diagnosis method based on CSF circulation is proposed in this invention. The outflow resistance of patients is calculated by monitoring various physiological parameters such as intracranial pressure during lumbar puncture and drainage, which can effectively improve the diagnostic accuracy of hydrocephalus, greatly shorten the diagnosis time of patients, and reduce the occurrence of misdiagnosis and missed diagnosis. At the same time, this technology can help doctors better understand the pathogenesis of hydrocephalus disease, so as to explore more effective treatment methods, and is expected to be applied to the diagnosis of congenital hydrocephalus, trauma, and subarachnoid hemorrhage after stimulating hydrocephalus and other related diseases and the formulation of personalized treatment strategies.

Finally, it should be noted that the above embodiment is only used to explain the technical scheme of the invention rather than to restrict it. Although the invention is described in detail concerning the better embodiment, ordinary technicians in this field should understand that they can still modify or replace the technical scheme of the invention, and these modifications or equivalent replacements cannot make the modified technical scheme out of the spirit and scope of the technical scheme of the invention.

What is claimed is:

1. A multi-modal data acquisition and hydrocephalus diagnosis method based on a cerebrospinal fluid (CSF) circulation, comprising the followings steps:
S1, a lumbar puncture;
S2, a perfusion test;
S3, a signal acquisition; and
S4, a data analysis.

2. The multi-modal data acquisition and hydrocephalus diagnosis method based on the CSF circulation according to claim 1, wherein in the step S1, a puncture point is selected at an intersection of a posterior superior iliac spine line and a posterior median line, corresponding to a 4-5 space of a lumbar spinous process, and after a skin of the puncture point is safely fixed, a lumbar puncture needle is inserted perpendicular to a back direction or slightly inclined to a head side.

3. The multi-modal data acquisition and hydrocephalus diagnosis method based on the CSF circulation according to claim 2, wherein after performing the lumbar puncture for a patient, the lumbar puncture needle is connected to a pressure sensor and a micro pump, and an intracranial pressure value of the patient at this time is continuously recorded for 5 min as an intracranial pressure (ICP) baseline.

4. The multi-modal data acquisition and hydrocephalus diagnosis method based on the CSF circulation according to claim 1, wherein in the step S2, a micro pump is started, and normal saline is injected into a lumbar intervertebral space at a constant speed of 60 mL/h, an intracranial pressure value and a plurality of physiological indicators of a patient are recorded and monitored in real-time through an intensive care monitoring plus (ICM+) until an intracranial pressure of the patient reaches a stable state, and the intracranial pressure value of the patient at this time is continuously recorded for 5 min as an IPC plateau.

5. The multi-modal data acquisition and hydrocephalus diagnosis method based on the CSF circulation according to claim 1, wherein in the step S3, the signal acquisition comprises a monitor, a medical pressure sensor, a blood oxygen probe, a five-lead electrocardiogram (ECG) monitoring, and an ICM+ multi-modal detection software.

6. The multi-modal data acquisition and hydrocephalus diagnosis method based on the CSF circulation according to claim 5, wherein the medical pressure sensor is configured to measure an intracranial pressure, the blood oxygen probe is configured to measure blood oxygen, the five-lead ECG monitoring is configured to measure a heart rate of a patient, and the monitor and the ICM+ multi-modal detection software are configured to monitor and record data of the patient in real-time.

7. The multi-modal data acquisition and hydrocephalus diagnosis method based on the CSF circulation according to claim 1, wherein the data analysis comprises the following steps:
S41, establishing a CSF circulation model;
S42, calculating a CSF outflow resistance;
S43, calculating an intracranial pressure pulse amplitude (AMP) and an elasticity coefficient Elasicity; and
S44, generating test reports for an auxiliary diagnosis.

8. The multi-modal data acquisition and hydrocephalus diagnosis method based on the CSF circulation according to claim 7, wherein in the step S41, an intracranial compliance denotes a volume compensation function in a cranial cavity, the intracranial compliance is a volume change caused by a change of a unit intracranial pressure, a calculation formula is as follows:
$$C = \frac{{dV}_{IC}}{{dP}_{IC}} \qquad ( 1 )$$
wherein C denotes the intracranial compliance, dV_{IC} denotes a change of an intracranial volume, and dP_{IC} denotes a change of an intracranial pressure;
when a volume of a cranial spinal cord gap increases, the intracranial compliance when the intracranial pressure is in a low-pressure state is much higher than the intracranial compliance when the intracranial pressure is in a high-pressure state, and the calculation formula of the intracranial compliance C is as follows:
$$C = \frac{1}{{KP}_{IC}} \qquad ( 2 )$$
wherein P_{IC} denotes the intracranial pressure, K denotes a specific elastic constant of a CSF circulatory system of a patient;
in a modeling of the CSF circulation, the calculation formula of the intracranial compliance C is as follows:
$$C = \frac{1}{K ( {P}_{IC} - {P}_{0} )} \qquad ( 3 )$$
wherein P₀ denotes a CSF constant storage pressure;
a model establishment comprises a whole circulation pathway of CSF in a human body, further comprises an infusion of normal saline, based on a law of conservation of a liquid, a total inflow of the liquid is equal to an outflow of the liquid, and an expression is as follows:
$${q}_{f} + {q}_{ex} = {q}_{a} + {q}_{s} \qquad ( 4 )$$
wherein q_{f} denotes a rate of a CSF formation, qₑₓ denotes a rate of a normal saline perfusion in subsequent perfusion tests, qₐ denotes a CSF pathway for a continuous circulation absorption, and qₛ denotes a CSF pathway stored in an intracranial support and protection of a brain;
a difference between the intracranial pressure and a dural venous sinus pressure P_{d} is calculated as follows:
$${q}_{a} = \frac{{P}_{IC} - {P}_{d}}{R} \qquad ( 5 )$$
wherein R denotes the CSF outflow resistance, P_{d} is the dural venous sinus pressure;
a change function of the intracranial pressure with time is obtained by substituting into formula (1) and formula (2):
$$\frac{{dP}_{IC} ( {V}_{IC} ( t ) )}{dt} = \frac{{dP}_{IC}}{{dV}_{IC}} \frac{{dV}_{IC}}{dt} = {KP}_{IC} \frac{{dV}_{IC}}{dt} \qquad ( 6 )$$
wherein t denotes the time, dt denotes a time change;
a cerebral blood flow is kept constant and a storage pathway of the CSF is calculated, a formula is as follows:
$${q}_{s} = \frac{{dV}_{IC}}{dt} \qquad ( 7 )$$
formula (6) and formula (4) are combined, a calculation formula is as follows:
$$\frac{{dP}_{IC}}{dt} = {KP}_{IC} {q}_{s} = {KP}_{IC} ( {q}_{f} + {q}_{ex} - {q}_{a} ) \qquad ( 8 )$$
a steady-state intracranial resting pressure Pᵣ is regulated by the dural venous sinus pressure, the CSF outflow resistance R, and a CSF formation rate q_{f}, and a calculation formula is as follows:
$${P}_{r} = {P}_{d} + {q}_{f} R \qquad ( 9 )$$
formula (8) and formula (5) are substituted into formula (9), a nonlinear differential equation of the CSF circulation model is obtained:
$$\frac{{dP}_{IC} ( t )}{dt} + {\frac{K}{R} [ {P}_{IC} ( t ) ]}^{2} - ( {Kq}_{ex} ( t ) + \frac{{KP}_{r}}{R} ) {P}_{IC} ( t ) = 0. \qquad ( 10 )$$

9. The multi-modal data acquisition and hydrocephalus diagnosis method based on the CSF circulation according to claim 8, wherein in the step S42, an integral factor method is used to solve formula (10), a formula is as follows:
$${P}_{IC} ( t ) = \frac{{e}^{\frac{K}{R} {\int }_{0}^{t} ( {Rq}_{ex} ( τ ) + {P}_{r} ) d τ}}{\frac{K}{R} {\int }_{0}^{t} {e}^{\frac{K}{R} {\int }_{0}^{t} ( {Rq}_{ex} ( τ ) + {P}_{r} ) d τ} + \frac{1}{{{P}_{IC}}^{( 0 )}}} \qquad ( 11 )$$
wherein τ is an independent variable in an integral process;
when the intracranial pressure gradually decreases from Pₚ to the steady-state intracranial resting pressure after P_{IC} rises to Pₚ through the perfusion test, an expression of a time relationship between the intracranial pressure and the normal saline perfusion is as follows:
$${P}_{IC} ( t ) = \frac{{p}_{P} {e}^{\frac{{KtP}_{r}}{R}}}{1 + \frac{{p}_{P}}{{p}_{r}} ( {e}^{\frac{{KtP}_{r}}{R}} - 1 )} \qquad ( 12 )$$
wherein p_{P} is a value of the intracranial pressure rising to a platform period;
under a condition of the steady-state intracranial resting pressure, P₀=0, the intracranial pressure P_{IC}(0) is a steady-state value, as Pᵣ, a perfusion speed is qₑₓ(t)=q_{inf}, and a calculation formula of P_{IC}(t) is as follows:
$${P}_{IC} ( t ) = \frac{{P}_{r} ( {P}_{r} + {Rq}_{inf} )}{{P}_{r} + {Rq}_{inf} {e}^{- \frac{K}{R} ( {P}_{r} + {Rq}_{inf} ) t}} \qquad ( 13 )$$
wherein q_{inf} denotes the perfusion speed;
under a condition of P₀≠0, the calculation formula of P_{IC}(t) is as follows:
$${P}_{IC} ( t ) = \frac{( {q}_{inf} + \frac{{P}_{r} - {P}_{0}}{R} ) ( {P}_{r} - {P}_{0} )}{\frac{{P}_{r} - {P}_{0}}{R} + {q}_{inf} [ {e}^{- K ( \frac{{P}_{r} - {P}_{0}}{R} + {q}_{inf} ) t} ]} + {P}_{0}$$
in a process of a constant pressure infusion test, dynamic data from a resting state to a steady state are collected and calculated, for a constant pressure infusion under a steady state condition, a differential equation is transformed into:
$${q}_{inf} = \frac{{P}_{IC} - {P}_{r}}{R}$$
wherein the CSF outflow resistance R is solved, and a calculation formula is as follows:
$$R = \frac{{P}_{level} - {P}_{r}}{{q}_{inf}} \qquad ( 16 )$$
wherein Pₗₑᵥₑₗ is a steady-state intracranial pressure produced by a constant pressure perfusion.

10. The multi-modal data acquisition and hydrocephalus diagnosis method based on the CSF circulation according to claim 7, wherein in the step S43, a frequency domain analysis and extraction of an intracranial pressure is carried out to obtain the intracranial pressure pulse amplitude, the intracranial pressure pulse amplitude is configured to reflect an increase of the intracranial pressure within a predetermined range, at the same time, the elasticity coefficient Elasicity is configured to represent a brain compliance, when the elasticity coefficient Elasicity is low, the brain compliance is poor.
